(19) United States
(12) Patent Application Publication
(10) Pub. No.: US 2005/0160501 A1
(43) Pub. Date: Jul. 21, 2005

(54) Genetic engineering of drought tolerance via a plastid genome

(76) Inventors: Henry Daniell, Winter Park, FL (US); Seung-Bum Lee, Orlando, FL (US); Myung Ok Byun, Orlando, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/010,133

(22) Filed: Dec. 10, 2004

Related U.S. Application Data
(63) Continuation of application No. 09/807,836, filed on Feb. 11, 2002, now abandoned.
(60) Provisional application No. 60/185,658, filed on Feb. 29, 2000.

(51) Int. Cl. A01H 1/00; C12N 15/82

(52) U.S. Cl. 800/289; 435/468

(57) ABSTRACT

This invention provides a method of conferring osmoprotection to plants. Plant plastid genomes, particularly the chloroplast genome, is transformed to express an osmoprotectant. The transgenic plants and their progeny display drought resistance. More importantly, such transgenic plants display no negative pleiotropic effects such as sterility or stunted growth.

### CROSS-REFERENCES TO RELATED APPLICATIONS

This patent application claims the benefit of U.S. Provisional Application No. 60/185,658, filed Feb. 29, 2000. This earlier provisional application is hereby incorporated by reference.

### STATEMENT REGARDING FEDERALLY SPONSORED RESEARCH

The work of this invention is support in part by the USDA-NRICGP grants 95-82770, 97-35504 and 98-0185 to Henry Daniell.

### FIELD OF INVENTION

This application pertains to the field of genetic engineering of plant plastid genomes, particularly chloroplasts and to methods of transforming plants to confer or increase drought tolerance and engineered plants which are drought tolerant.

### DESCRIPTION OF RELATED ART

Patents of Interest

Londesborough et. al., in U.S. patent No. 5,792,921 (1998), entitled “Increasing the trehalose content of organisms by transforming them with combinations of the structural genes for trehalose synthase,” and U.S. Pat. No. 6,130,368 (2000), entitled “Transgenic plants producing trehalose”, proposed a method for increasing trehalose content in various organisms through nuclear transformation.

Hoekema, in U.S. Pat. No. 5,925,804 (1999), entitled “Production of Trehalose in Plants,” proposes a method of engineering plants to produce trehalose. This patent suggests the transformation of plants by introducing to the plant nuclear genome any trehalose phosphate synthase gene driven by an appropriate promoter.

Strom, et al., in U.S. Pat. No. 6,133,038 entitled “Methods and compositions related to the production of trehalose” (2000), described the genes involved in the biosynthesis of trehalose, trehalose synthase and trehalose-6-phosphate. Methods for producing trehalose biosynthetic enzymes in a host cell through transformation of the cell's nucleus are also proposed. In addition, the patent also suggests nuclear transgenic host cells which contain recomvinant DNA constructs encoding for a trehalose synthase, trehalose phosphatase or both trehalose synthase and, trehalose phosphatase.

### BACKGROUND OF THE INVENTION

Effects of Increased Trehalose Accumulation

Water stress due to drought, salinity or freezing is a major limiting factor in plant growth and development. Trehalose is a non-reducing disaccharide of glucose and its synthesis is mediated by the trehalose-6-phosphate (T6P) synthase and trehalose-6-phosphate phosphatase complex in Saccharomyces cerevisiae. In S. cerevisiae, this complex consists of at least three subunits performing either T6P synthase (TPS1), T6P phosphatase (TPS2) or regulatory activities (TPS3 or TSLI). Trehalose is found in diverse organisms including algae, bacteria, insects, yeast, fungi, animal and plants. Because of its accumulation under various stress conditions such as freezing, heat, salt or drought, there is general consensus that trehalose protects against damages imposed by these stresses. Trehalose is also known to accumulate in anhydrobiotic organisms that survive complete dehydration, the resurrection plant and some desiccation tolerant angiosperms. Trehalose, even when present in low concentrations, stabilizes proteins and membrane structures under stress because of the glass transition temperature, greater flexibility and chemical stability/inertness.

Prior Efforts to Engineer Plants for Trehalose Production

There have been several efforts to generate various stress resistant transgenic plants by introducing gene(s) responsible for trehalose biosynthesis, regulation or degradation. When trehalose accumulation was increased in transgenic tobacco plants by over-expression of the yeast TPS1, trehalose accumulation resulted in the loss of apical dominance, stunted growth, lancet-shaped leaves and some sterility. Altered phenotype was always correlated with drought tolerance, plants showing severe morphological alterations had the highest tolerance under stress conditions.

Advantages of Transforming Plants Through the Chloroplast

In order to minimize the pleiotropic effects observed in the nuclear transgenic plants accumulating trehalose, this invention compartmentalizes trehalose accumulation within chloroplasts. Several toxic compounds expressed in transgenic plants have been compartmentalized in chloroplasts, even through no targeting sequence was provided indicating that this organelle could be used as a repository like the vacuole. Also, osmoprotectants are known to accumulate inside chloroplasts under stress conditions. Inhibition of trehalase activity is known to enhance trehalose accumulation in plants. Therefore, trehalose accumulation in chloroplast may be protected from trehalase activity in the cytosol, if trehalase was absent in the chloroplast.

In addition, chloroplast transformation has several other advantages over nuclear transformation. A common environmental concern about nuclear transgenic plants is the escape of foreign genes through pollen or seed dispersal, thereby creating super weeds or causing genetic pollution among other crops. The latter has resulted in several lawsuits and shrunk the European market for organic produce from Canada from 83 tons in 1994-1995 to 20 tons in 1997-1998. These are serious environmental concerns, especially when plants are genetically engineered for drought tolerance, because of the possibility of creating robust drought tolerant weeds and passing on undesired pleiotropic traits to related crops. Chloroplast transformation should also overcome some of the disadvantages of nuclear transformation that result in lower levels of foreign gene expression, such as gene suppression by positional effect or gene silencing.

Chloroplast genetic engineering has been successfully employed to address aforementioned concerns. For example, chloroplast transgenic plants expressed very high level of insect resistance, due to expression of 10,000 copies of foreign genes per cell, thereby overcoming the problem of insect resistance observed in nuclear transgenic plants. Similarly, chloroplast derived herbicide resistance overcomes out-cross problems of nuclear transgenic plants because of maternal inheritance of plastid genomes. This invention thus presents a solution to the pitfalls of nuclear expression of TPS1 in transgenic plants.

Non-Obvious Nature of the Invention.

Trehalose is a non-reducing disaccharide of glucose and is found in diverse organisms including algae, bacteria, insects, yeast, fungi, animal and plants. Because of its accumulation under various stress conditions such as freezing, heat, salt or drought, there is general consensus that trehalose protects against damages imposed by these stresses. Trehalose is also known to accumulate in anhydrobiotic organisms that survive complete dehydration, the resurrection plant and some desiccation tolerant angiosperms.

There have been several efforts to generate various stress resistant transgenic plants by introducing gene(s) responsible for trehalose biosynthesis, regulation or degradation. When trehalose accumulation was increased in nuclear transgenic tobacco plants by over-expression of the yeast TPS1, trehalose accumulation resulted in the loss of apical dominance, stunted growth, lancet shaped leaves and some sterility. Altered phenotype was always correlated with drought tolerance; plants showing severe morphological alterations had the highest tolerance under stress conditions. Prior to this invention, it was not obvious that accumulation of trehalose within plastids would minimize the pleiotropic effects observed in the nuclear transgenic plants accumulating trehalose or damage plastids. There were no prior reports of trehalose accumulation within plastids or localization of enzymes of trehalose biosynthetic pathway within plastids.

Osmoprotectants are known to accumulate inside chloroplasts under stress conditions but their mode of action is to provide osmotic protection by accumulation of such compounds (as sugars or amino acids) in large quantities. This invention, demonstrates that the protection is offered by accumulation of small quantities of trehalose which was not adequate to provide protection from dehydration but rather stability of biological membranes. Inhibition of trehalase activity is known to enhance trehalose accumulation in the cytosol but there are no reports of the presence or absence of trehalase within plastids. Therefore, it was unanticipated that trehalose accumulation within plastids would be protected from trehalase activity. Prior to this invention, there were no reports of using plastid transformation as a strategy to confer drought tolerance to transgenic plants.

### BRIEF SUMMARY OF THE INVENTION

This invention provides a method to transform plants through the plastids, particularly chloroplasts, to confer drought tolerance to plants. The vectors with which to accomplish the chloroplast transformation is provided. The transformed plants and their progeny are provided. The transformed plants and their progeny display drought resistance. More importantly, they display no negative pleiotropic effects such as sterility or stunted growth.

The present invention is applicable to all plastids of plants. These include chromoplasts which are present in the fruits, vegetables and flowers; amyloplasts which are present in tubers like the potato; proplastids in roots; leucoplasts and etioplasts, both of which are present in non-green parts of plants.

The present invention provides a method to increase water stress tolerance in dicotyledonous or a monocotyledonous plant, comprising introducing an expression cassette into the cells of a plant to yield transformed plant cells. Plant cells include cells of monocotyledenous plants such as cereals, including corn (Zea mays), wheat, oats, rice, barley, millet and cells of dicotyledenous plant such as soybeans and vegetables like peas. The expression cassette comprises a preselected DNA sequence encoding an enzyme which catalyzes the synthesis of an osmoprotectant, operably linked to a promoter functional in the chloroplast plant cell. The enzyme encoded by the DNA sequence is expressed in the transformed plant cells to increase the level of osmoprotection so as to render the transformed cells substantially tolerant or resistant to a reduction in water availability that inhibits the growth of untransformed cells of the plant.

As used herein, an “osmoprotectant” is an osmotically active molecule which, when that molecule is present in an effective amount in a cell or plant, confers water stress tolerance or resistance, or salt stress tolerance or resistance, to the cell or plant; when present in lower amounts in a cell or plant, an “osmoprotectant” confers membrane stability. Those skilled in the art will appreciate that an osmoprotectant confers resistance to water or salt stress when present in the cell in high amounts, and confers membrane stability in lower amounts. Osmoprotectants include sugars such as monosaccharides, disaccharides, oligosaccharides, polysaccharides, sugar alcohols, and sugar derivatives, as well as proline and glycine-betaine. A preferred embodiment of the invention is an osmoprotectant that is a sugar. Useful osmoprotectants include fructose, erythritol, sorbitol, dulcitol, glucoglycerol, sucrose, stachyose, raffinose, ononitol, mannitol, inositol, methyl-inositol, galactol, hepitol, ribitol, xylitol, arabitol, trehalose, and pinitol.

Genes which encode an enzyme that catalyzes the synthesis of an osmoprotectant include genes encoding mannitol dehydrogenase (Lee and Saier, J. Bacteriol., 153 (1982)) and trehalose-6-phosphate synthase (Kaasen et al., J. Bacteriol., 174, 889 (1992)). Through the subsequent action of native phosphatases in the cell or by the introduction and coexpression of a specific phosphatase into the nucleus, these introduced genes result in the accumulation of either mannitol or trehalose in the nucleus, respectively, both of which have been well documented as protective compounds able to mitigate the effects of stress. Mannitol accumulation in the nucleus of transgenic tobacco has been verified and preliminary results indicate that plants expressing high levels of this metabolite are able to tolerate an applied osmotic stress (Tarczynski et al., cited supra (1992), (1993)).

Also provided is an isolated transformed plant cell and an isolated transformed plant comprising said transformed cells, which cell and plant are substantially tolerant of or resistant to a reduction in water availability. The cells of the transformed monocot plant comprise a recombinant DNA sequence comprising a preselected DNA sequence encoding an enzyme which catalyzes the synthesis of an osmoprotectant. The preselected DNA sequence is present in the cells of the transformed plant and the enzyme encoded by the preselected DNA sequence is expressed in those cells to yield an amount of osmoprotectant effective to confer tolerance or resistance to those cells to a reduction in water availability that inhibits the growth of the corresponding untransformed plant cells. A preferred embodiment of the invention includes a transformed plant that has an improved osmotic potential when the total water potential of the transformed plant approaches zero relative to the osmotic potential of a corresponding untransformed plant.

As used herein, a “preselected” DNA sequence is an exogenous or recombinant DNA sequence that encodes an enzyme which catalyzes the synthesis of an osmoprotectant, such as sugar. The enzyme preferably utilizes a substrate that is abundant in the plant cell. It is also preferred that the preselected DNA sequence encode an enzyme that is active without a co-factor, or with a readily available co-factor. For example, the mild gene of E. Coli encodes a mannitol-1-phosphate dehydrogenase (M1PD). The only co-factor necessary for the enzymatic activity of M1PD in plants is NADH and the substrate for M1PD in plants is fructose-6-phosphate. Both NADH and fructose-6-phosphate are plentiful in higher plant cells.

As used herein, “substantially increased” or “elevated” levels of an osmoprotectant in a transformed plant cell, plant tissue, plant part, or plant, are greater than the levels in an untransformed plant cell, plant part, plant tissue, or plant, i.e., one where the chloroplast genome has not been altered by the presence of a preselected DNA sequence. In the alternative, “substantially increased” or “elevated” levels of an osmoprotectant in a water-stressed transformed plant cell, plant tissue, plant part, or plant, are levels that are at least about 1.1 to 50 times; preferably at least about 2 to 30 times, and more preferably about 5-20 times, greater than the levels in a non-water-stressed transformed plant cell, plant tissue, plant part of plant.

As used herein, a plant cell, plant part, plant tissue or plant that is “substantially resistant or tolerant” to a reduction in water availability is a plant cell, plant part, plant tissue, or plant that grows under water-stress conditions, e.g., high salt, low temperatures, or decreased water availability, that normally inhibit the growth of the untransformed plant cell, plant tissue, plant part, or plant, as determined by methodologies known to the art. Methodologies to determine plant growth or response to stress include, but are not limited to, height measurements, weight measurements, leaf area, plant water relations, ability to flower, ability to generate progeny, and yield. For example, a stably transformed plant of the invention has a superior osmotic potential during a water deficit relative to the corresponding.

As used herein, an “exogenous” gene or “recombinant” DNA is a DNA sequence that has been isolated from a cell, purified, and amplified.

As used herein, the term “isolated” means either physically isolated from the cell or synthesized in vitro in the basis of the sequence of an isolated DNA segment.

As used herein, a “native” gene means a DNA sequence or segment that has not been manipulated in vitro, i.e., has not been isolated, purified, and amplified.

The invention also provides, preferably, a plastid vector that is capable of stably transforming and conferring drought resistance to tolerance to different plant species.

The invention provides a plastid vector comprising of a DNA construct. The DNA construct includes a 5′ part of the plastid DNA sequence inclusive of a spacer sequence; a promoter that is operative in the plastid; heterologous DNA sequences comprising at least one gene of interest encoding a molecule; a gene that confers resistance to a selectable marker; a transcription termination region functional in the target plant cells; and a 3′ part of the plastid DNA sequence inclusive of a spacer sequence. The molecule can be a peptide of interest. Preferably, the vector includes a ribosome binding site (rbs) and a 5′ untranslated region (5′UTR). A promoter functional in green or non-green plastids is used in conjunction with the 5′UTR.

Further, the invention provides a heterologous DNA sequence, which codes for an osmoprotectant, such as the Yeast T6P synthase gene (TSP1 gene), the E. coli otsA gene. The invention also provides the psbA 3′ region, which enhances the translation of foreign genes.

The invention provides a promoter is one that is operative in green and non-green plastids such as the 16SrRNA promoter, the psbA promoter, and the accD promoter.

The invention provides a gene that confers resistance, such as antibiotic resistance like the aadA gene or an antibiotic-free selectable marker such as BADH or the chlB gene, as a selectable marker.

All known methods of transformation can be used to introduce the vectors of this invention into target plant plastids including bombardment, PEG Treatment, Agrobacterium, microinjection, etc.

The invention provides transformed crops, like solanaceous plants that are either monocotyledonous or dicotyledonous. Preferably, the plants are those having economic value which are edible for mammals, including humans.

Any plant can be transformed to an osmprotectant-expressing plant in accordance of the inyention which can carry a helogerous DNA sequence which encodes a desired trait. The transformed osmoprotectant-expressing plant need not comprise such a trait other than the DNA sequence which encodes the osmoprotentant.

The invention provides plants that have been transformed via the chloroplast which accumulate trehalose at an amount at least 17-fold higher than non-transformed plants which are drought resistant.

The invention provides plants that have been transformed via the chloroplast which has at least a seven-fold increase in TPS1 activity.

The invention provides plants that have been transformed via the chloroplast which, in the T₀ generation, display otherwise normal phenotype other than decreased growth and delayed flowing. The invention further provides that the T₁/T₂ generations of the transformed plants display no pleiotropic effects.

The invention provides the transformed chloroplasts of the target plants which contain high levels of trehalose.

The invention provides for chloroplast transformant seedlings which are drought resistant which are resistant to medium containing 3% to 6% PEG.

The invention provides a method to confer drought resistance to plants via chloroplast transformation with a universal chloroplast vector which contains a drought-resistant or osmoprotectant gene and the accumulation of high levels of trehalose in the chloroplast.

The invention provides a method to transform a target plant for expression of the TPS1 gene leading to accumulations of trehalose in the chloroplast of the plant cells and eliminating adverse pleiotropic effects.

The invention provides proof of integration of the heterologous DNA sequence into the chloroplast genome by PCR.

The invention provides an environmental friendly method of engineering drought resistance to plants through chloroplast transformation.

Yeast trehalose phosphate synthase (TPS1) gene was introduced into the tobacco chloroplast or nuclear genomes to study resultant phenotypes. PCR and Southern blots confirmed stable integration of TPS1 into the chloroplast genomes of T₁, T₂ and T₃ transgenic plants. Northern blot analysis of transgenic plants showed that the chloroplast transformant expressed 16,966-fold more TPS1 transcript than the best surviving nuclear transgenic plant. Although both the chloroplast and nuclear transgenic plants showed significant TPS1 enzyme activity, no significant trehalose accumulation was observed in T₀/T₁ nuclear transgenic plants whereas chloroplast transgenic plants showed 15-25 fold higher accumulation of trehalose than the best surviving nuclear transgenic plants. Nuclear transgenic plants (T₀) that showed significant amounts of trehalose accumulation showed stunted phenotype, sterility and other pleiotropic effects whereas chloroplast transgenic plants (T₁, T₂, T₃) showed normal growth and no pleiotropic effects. Chloroplast transgenic plants also showed a high degree of drought tolerance as evidenced by growth in 6% polyethylene glycol whereas untransformed plants were bleached. After 7 hr drying, chloroplast transgenic seedlings (T₁, T₃) successfully rehydrated while control plants died. There was no difference between control and transgenic plants in water loss during dehydration but dehydrated leaves from transgenic plants (not watered for 24 days) recovered upon rehydration while control leaves died. In order to prevent escape of drought tolerance trait to weeds and associated pleiotropic traits to related crops, it is desirable to genetically engineer crop plants for drought tolerance via the chloroplast genome instead of the nuclear genome.

### BRIEF DESCRIPTION OF THE DRAWINGS

FIG. 1. PCR analysis of control and chloroplast transformants. A. Map of pCt-TPS1, chloroplast transformation vector and primer landing sites. P denotes plus strand and M denotes minus strand. Please note that tRNA genes contain introns. B. 1% agarose gel containing PCR products using total plant DNA as template. M: 1 kb ladder; 1. N. Nicotiana tabacum Burley, untransformed control; Lanes 1, 3, 5: pCt basic vector transformants. 2, 4, 6: pCt-TPS1 transformants. C. Map of the nuclear expression vector pHGTPS1.

FIG. 2. Southern blot analysis of control, T₁ and T₃ chloroplast transgenic plants. A. Site of integration of foreign genes into the chloroplast genome and expected fragment sizes in Southern blots. P1 is the 0.81 kb BamHI-Bg1II fragment containing chloroplast DNA flanking sequences used for homologous recombination. P2 is the 1.5 kb Xba1 Fragment containing the TPS1 coding sequence. B. Southern blot of DNA digested with Bg1II and hybridized with probes P1 or P2. Lanes: C, untransformed control; 1, T₁ generation chloroplast transformant; 2, T₃ generation chloroplast transformant.

FIG. 3. Northern and western blot analyses of control, nuclear and chloroplast transgenic plants. A, D Western blots detected through chemiluminescence (100 μg total protein per lane). B, E Northern blots detected using ³²P TPS1 probe. C, F Ethidium bromide stained RNA gel before blotting (10 μg total RNA loaded per lane). Panel A, B, C: T₀ nuclear and T₁ chloroplast transgenic plants. Lanes: 1. N. t. xanthi control; 2˜5: T₀ nuclear transgenic plants. 2, X-113; 3. X-119; 4. X-121; 5. X-224; 6: N.t. Burley control; 7: chloroplast transgenic plant (T₁). Panel D, E, F: T₁ nuclear and T₂ chloroplast transgenic plants. Lanes: 1. N t xanthi control; 2, 3: T₁ nuclear transgenic plants 2, X-113; 3.X-119; 4: Nt. Burley control; 5: chloroplast transgenic plant (T₂).

FIG. 4. Nuclear and chloroplast transgenic plants to illustrate pleiotropic effects. 1. N. t xanthi control; 2˜5: T₀ nuclear transgenic plants 2, X-113; 3.X-121; 4. X-119; 5. X-224; 6, T₁ chloroplast transgenic plant; 7, N. t. Burley control.

FIG. 5. Germination of T₁, T₂ and T₃ generation of chloroplast transformants and untransformed control on MS plate containing spectinomycin (500 μg/ml).

FIG. 6. Assay for drought tolerance on PEG. Four week old seedlings on MS medium containing 3% (A, B) or 6% (C, D) polyethylene glycol (MW 8,000). A, C: Control untransformed N.t. Burley. B, D: T₁ Chloroplast transgenic plants.

FIG. 7. Dehydration/rehydration assay. Three week old seedlings from control and chloroplast transgenic lines germinated on agarose in the absence or presence of spectinomycin (500 μg/ml) were air-dried at room temperature in 50% relative humidity. After 7 hrs drying, seedlings were rehydrated for 48 hrs by placing roots in MS medium. A, untransformed; B,C, T₁ and T₃ chloroplast transgenic lines.

FIG. 8. Water loss assay. Detached leaves from mature plants at similar developmental stages were dried at room temperature in 25% relative humidity. Leafweight during drying was recorded and shown as percentage of initial fresh weight.

FIG. 9. Dehydration and rehydration of potted plants. Potted plants were not watered for 24 days and rehydrated for 24 hours. Arrows indicate fully dried leaves that either recovered or did not recover from dehydration. A, C: Control untransformed; B,D: chloroplast transgenic plants.

### DETAILED DESCRIPTION OF THE INVENTION

This invention discloses a method of conferring drought tolerance to plants by transforming plants via the chloroplast with a vector that contains a DNA sequence encoding a gene of interest that protects against water stress. In the preferred embodiment of this invention, the vector used is the universal vector as described by Daniell in WO99/10513, which is incorporated herein by reference. Other vectors that are capable of chloroplast transformation such as pUC, pBR322, pBlueScript, pGem and others described in U.S. Pat. Nos. 5,693,507 and 5,932,479 may be used. In the preferred embodiment of this invention, the osmoprotection is the yeast trehalose-6-phosphate synthase (TSP1). Other genes which are capable of conferring drought resistance or osmoprotection may also be used.

Expression of Yeast TPS1 in E. coli:

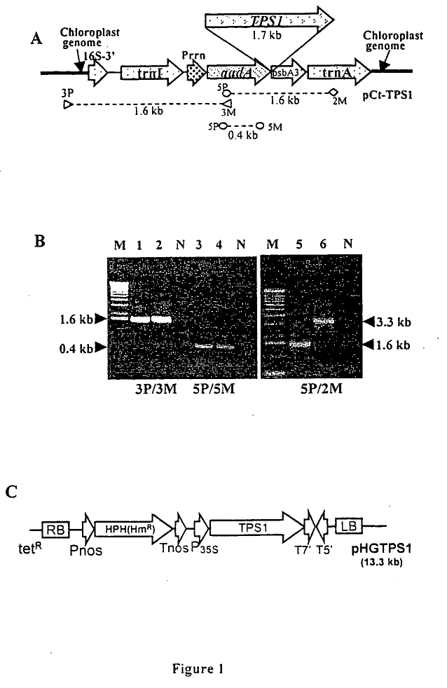

It is known that the yeast trehalose-6-phosphate synthase gene can be expressed in nuclear transgenic plants. Because chloroplasts are prokaryotic in nature, it is desirable to test expression levels of the eukaryotic yeast TPS1 gene in E coli. Because of the high similarity in the transcription and translation systems between E. coli and chloroplasts, expression vectors are routinely tested in E. coli before proceeding with chloroplast transformation of higher plants. Therefore, the TPS1 gene from yeast was cloned into the E. coli expression vector pQE 30 (see FIG. 1A for details of pQE-TPS1) and expressed in a suitable E. coli strain M15 (pREP4). SDS-PAGE as shown in FIG. 1B shows the presence of TPS1 protein in crude cell extracts, even with Coomassie Blue stain (lane 1), indicating high levels of expression. Western blot analysis using TPS1-antibody confirms the true identity of the expressed protein as shown in FIG. 1B, lane 41. These results confirm that the codon preference of TPS1 is compatible for expression in a prokaryotic compartment. Hyper-expression also facilitated purification as shown in FIG. 1, lanes 2.55 and preparation of polyclonal antibody for characterization of transgenic plants.

Chloroplast and Nuclear Expression Vectors.

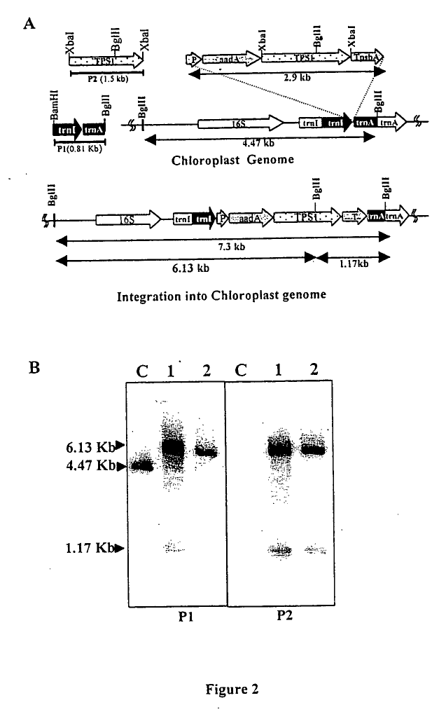

Having confirmed suitability for prokaryotic expression, the yeast TPS1 gene was inserted into the universal chloroplast expression vector pCt-TPS1 as shown in FIG. 2B. This vector can be used to transform chloroplast genomes of several plant species because the flanking sequences are highly conserved among higher plants. This vector contains the 16SrRNA promoter (Prrn) driving the aadA (aminoglycoside 3″-adenylyl transferase) and TPS1 genes with the psbA 3′ region (the terminator from a gene coding for photosystem II reaction center component) from the tobacco chloroplast genome. It is known that the 16SrRNA promoter is one of the strong chloroplast promoters and the psbA 3′ region stabilized transcripts to avoid hyper-expression of TPS-1 and associated Pleiotropic effects. The yeast ribosme binding site (RBS) was used instead of the genome 26 chloroplast RBS (GGAGG). This construct integrates both genes into the spacer region between the chloroplast transfer RNA genes coding for alanine and isoleucine within the inverted repeat (IR) region of the chloroplast genome by homologous recombination. For nuclear expression, the yeast TPS1 gene was inserted into the binary vector pHGTPS1 (FIG. 2C), in which the TPS1 gene is driven by the CaMV 35S promoter and the hph gene is driven by the nopaline synthase promoter. The expression cassette is flanked by both the left and right T-DNA border sequences.

The binary vector pHGTPS1 was mobilized into the Agrobacterium tumafaciens strain LBA 4404 by electroporation. Transformed Agrobacterium strain was introduced into Nicotiana tabaccum var xanthi using the leaf disc transformation method. Ninety two independent TPS1 nuclear tranformants were obtained on hygromycin selection. Seventeen confirmed nuclear tranformants were analyzed by northern blots. Among tranformants showing various levels of transcripts, five tranformants with strong, moderate, weak, very weak and absence of transcripts were chosen for further characterization. For chloroplast transformation, green leaves of N. tabacum var. Burley were transformed with the chloroplast integration and expression vector by the biolistic process. Bombarded leaf segments were selected on spectinomycin/streptomycin selection medium. Integration of foreign gene into the chloroplast genome was determined by PCR screening of chloroplast tranformants, (FIG. 2A). Primers were designed to eliminate mutants, nuclear integration and to determine whether the integration of foreign genes had occurred in the chloroplast genome at the directed site by homologous recombination. Primers 5P/5M land within the aadA gene and should generate a 0.4 kbp fragment if the aadA gene was present in transgenic plants and eliminates the possibility of mutation that could otherwise confer streptomycin/spectinomycin resistance. FIG. 2A shows the presence of 0.4 kbp PCR product in plants transformed with the universal vector alone (pCt) or the universal vector containing the TPS1 gene (pCt-TPS1), but not in control untransformed plants, confirming that these are transgenic plants and not mutants. The strategy to distinguish between nuclear and chloroplast transgenic plants was to land one primer (3P) on the native chloroplast genome adjacent to the point of integration and the second primer (3M) on the aadA gene. This primer set generated 1.6 kbp PCR product in chloroplast tranformants obtained with the universal vector (pCt) and the universal vector containing the TPS1 gene (pCt-TPS1). Because this product can not be obtained in nuclear transgenic plants, the possibility of nuclear integration can be eliminated. Another primer set was designed to test integration of the entire gene cassette. The presence of the expected size PCR products using 5P/5M confirms that the entire gene cassette has been integrated and that there has been no internal deletions or loop outs during integration via homologous recombination.

Determination of Chloroplast Integration, Homoplasmy and Copy Number:

Since there are no significant differences in the level of foreign gene expression among different chloroplast transgenic lines, one line was chosen to generate subsequent generations (T₁T₂T₃). Southern blot analysis was performed using total DNA isolated from transgenic and wild type tobacco leaves. Total DNA was digested with a suitable restriction enzyme. Presence of a Bg1II at the 3′ end of the flanking 16S rRNA gene and the trnA intron allowed excision of predicted size fragments in the chloroplast tranformants and untransformed plants. To confirm foreign gene integration and homoplasmy, individual blots were probed with the chloroplast DNA flanking sequence (probe P1, FIG. 2A). In the case of the TPS1 integrated plastid tranformants (T₁T₂), the 6 border sequence hybridized with 6.13 and 1.17 kbp fragments while it hybridized with a native 4.47 kbp fragment in the untransformed plants (FIG. 2B). The copy number of the integrated TPS1 gene was also determined by establishing homoplasmy in transgenic plants. Tobacco chloroplasts contain about 10,000 copies of chloroplast genomes per cell. If only a fraction of the genomes were transformed, the copy number should be less than 10,000. By confirming that the TPS1 integrated genome is the only one present in transgenic plants, one could establish that the TPS1 gene copy number could be as many as 10,000 per cell.

DNA gel blots were also probed with the TPS1 gene coding sequence (probe P2) to confirm integration into the chloroplast genomes. In chloroplast transgenic plants (T₁T₃), the TPS1 gene coding sequence hybridized with 6.13 and 1.17 kbp fragments which also hybridized with the border sequence in plastid transgenic lines (FIG. 2B). This confirms that the tobacco tranformants indeed integrated the intact gene expression cassette into the chloroplast genome and that there has been no internal deletions or loop out during integration via homologous recombination.

Analysis of Transcript Level in Nuclear and Chloroplast Tranformants:

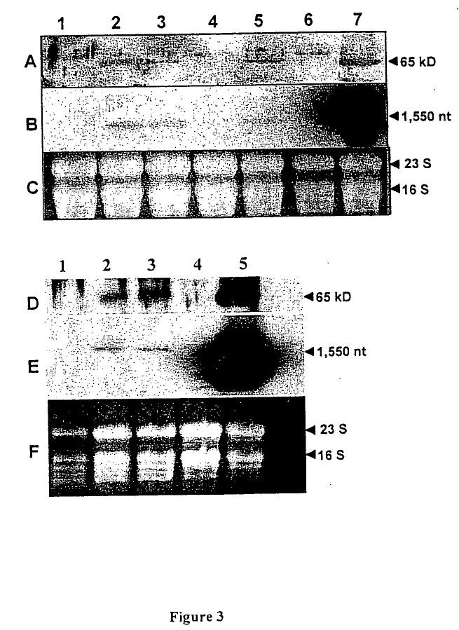

For comparison of introduced gene expression between chloroplast and nuclear tranformants, northern blot analysis of transgenic tobacco at similar developmental stages was performed in T₁, T, and T₂ plants. As shown in FIG. 3, quantification of transcription level showed that the chloroplast transformant (T2) expressed 16,960-fold (FIG. 3E, lane 5) more TPS1 transcript than that of highly expressing nuclear (T₁) transformant (FIG. 3E, lanes 2, 3). Similar results were obtained when T₁ chloroplast (FIG. 3B, lane 7) and T₀ nuclear transgenic plants (FIG. 3B, lanes 2˜5) were compared. This large difference in TPS1 expression between nuclear and chloroplast transgenic plants should be due to the presence of thousands of TPS1 gene copies in each cell of transgenic tobacco. FIG. 3 (C, F) show ethidium bromide stained RNA gels before blotting; this confirms that equal amount of RNA (10 μg) was loaded in all lanes. It is remarkable that the 16SrRNA promoter is driving both genes very efficiently, eliminating the need for inserting additional promoters for the gene of interest.

Western Blot Analysis of Nuclear and Chloroplast Tranformants:

Polyclonal antibodies raised against the TPS1 protein overexpressed and purified from E. coli (see experimental protocol) were used for immunoblotting (FIG. 3A, D). A 60 kDa TPS1 polypeptide was detected in the T₀ nuclear (FIG. 3A, lanes 2,3,5), T₁ nuclear (3D lanes 2,3) and T₁ plastid (FIG. 3A, lane 7) and T₂ plastid (FIG. 3D, lane 0.5) tranformants. However, no TPS1 was detected in the untransformed control (FIG. 3A, lanes 1,6; 3D 1,4)) and transgenic plants which showed no TPS1 transcript (FIG. 3A, lane 4). As anticipated, western blots showed only a five or ten fold increase in TPS1 protein in chloroplast over highly expressing nuclear transgenic plants. This is because of the fact that the chloroplast vector pCt-TPS1 was intentionally designed to lower translation by not inserting a chloroplast preferred ribosome binding site (GGAGG), so that transgenic plants are not killed by hyper-expression of TPS1. This level expression was adequate to compare trehalose accumulation in cytosolic and chloroplast compartments and observe resultant phenotypic/physiological changes. T₁ nuclear and T₂ chloroplast transgenic plants had higher levels of TPS1 protein; this may be due to homozygous TPS1 alleles or homoplasmy.

Quantification of Trehalose-6-Phosphate and Trehalose in Tranformants:

Trehalose formation is a two step process, involving trehalose-6-phosphate synthase and trehalose 6-phosphate phosphatase. Trehalose-6-phosphate was not detected in all tested chloroplast and nuclear transformers even though the TPS2, trehalose-6-phosphate phosphatase that converts T6P to trehalose, was not introduced (Table 1). Conversion of T6P to trehalose should have been accomplished by endogenous tobacco trehalose phosphatase or by any non-specific endogenous phosphatase. Simultaneous expression of both enzymes in transgenic plants resulted only in marginal increase of trehalose accumulation in previous studies, confirming that it is adequate to express only TPS1. Leaf extracts from both nuclear and chloroplast transgenic plants catalyzed the synthesis of trehalose 6-phosphate from glucose-6-phosphate and UDP-glucose whereas untransformed tobacco had very low activity. T₀ Chloroplast and nuclear transgenic plants showed a 7-10 fold higher TPS1 activity than untransformed control plants. The amount of trehalose present in untransformed control plants and T₀ nuclear transgenic plants were similar whereas chloroplast transgenic plants accumulated a 17-25 fold mm trehalose than the best surviving nuclear transgenic plants (Table 0.1). T₁ nuclear transgenic plants accumulated less trehalose than control untransformed plants whereas T₁ chloroplast transgenic plants continued to accumulate high levels of trehalose (Table 1). Observation of comparable TPS1 activity in both nuclear and chloroplast transgenic plants but lack of trehalose accumulation in nuclear transgenic planes indicates that trehalose may be degraded in the cytosol by trehalase but not in the chloroplast compartment. This is consistent with previous studies on inhibition of trehalase activity that resulted in trehalose accumulation in the cytosol.

Drought Tolerance and Pleiotropic Effects:

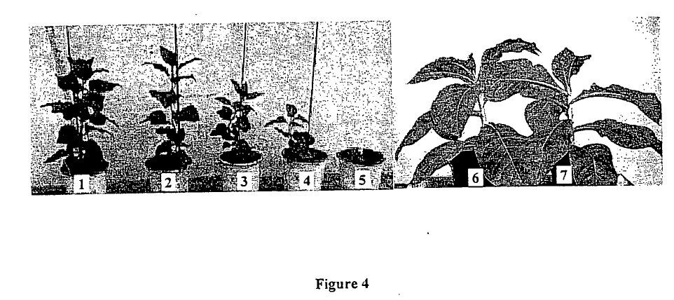

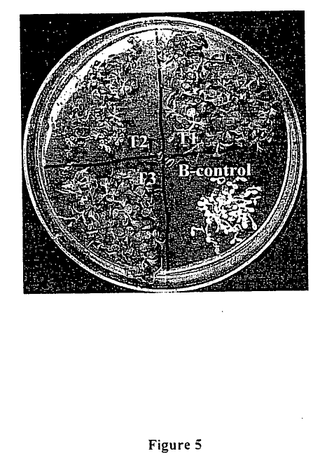

Chloroplast and nuclear tranformants were examined for drought tolerance and pleiotropic 6 effects. After six weeks of growth in vitro, rooted shoots were transferred to pots and grown in the greenhouse. TPS1 nuclear tranformants showed moderate to severe growth retardation, lancet-shaped leaves and infertility (FIG. 4). The chloroplast tranformants (T₀) showed decreased growth rate and delayed flowering but all subsequent generations (T₁, T₂) showed similar growth rates and fertility as controls. The nuclear transgenic lines of stunted phenotype showed delayed flowering and produced fewer seeds compared to wild type or did not flower. This result is consistent with prior observations which demonstrated that E. coli otsA (TPS1) and S. cerevisiae TPS1 transgenic plants exhibited stunted plant growth and other pleiotropic effects. The nuclear transgenic line showing severe growth retardation did not flower. T₁ nuclear transgenic plants that survived showed no growth retardation and trehalose accumulation. Therefore, these plants could not be used for appropriate comparison with chloroplast transgenic plants. When the seeds of chloroplast transgenic plant (crossed between transgenic female and untransformed male) and wild type seeds were germinated on MS medium containing spectinomycin, all chloroplast transgenic progeny were spectinomycin resistant while all wild type seedlings were sensitive to spectinomycin (FIG. 5).

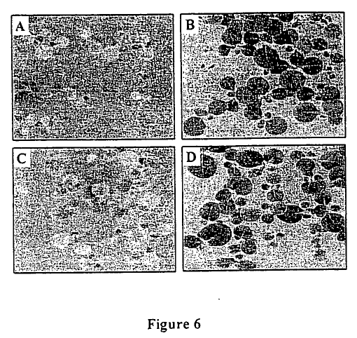

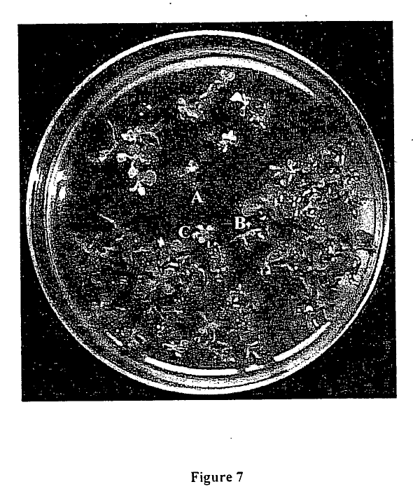

Because TPS1 transgenic lines showed accumulation of trehalose, they were tested for drought tolerance. Seeds of chloroplast and nuclear transgenic plants were germinated on the MS medium containing polyethylene glycol. As shown in FIG. 6, chloroplast transformant seedlings showed resistance to medium containing 3% and 6% PEG whereas control and nuclear transgenic seedlings exhibited severe dehydration, necrosis and severe growth retardation, ultimately resulting in death. Three-week-old seedlings were chosen to study drought tolerance by dehydration and subsequent rehydration. When seedlings were dried for 7 hours at room temperature in 50% relative humidity, they were all affected by dehydration. However, when dehydrated seedlings were rehydrated for 48 hours in MS medium, all chloroplast transgenic lines recovered while all control seedlings were bleached (FIG. 7). Even the couple of control seedlings that partly survived (because of uneven drying of seedlings on filter papers) eventually died. These results suggest that the loss of water from TPS1 transgenic plants may not be decreased but the ability to recover from drought was dramatically enhanced. This is consistent with existing understanding that trehalose functions by protecting biological membranes rather than regulating water potential (Iwahashi et al., 1995).

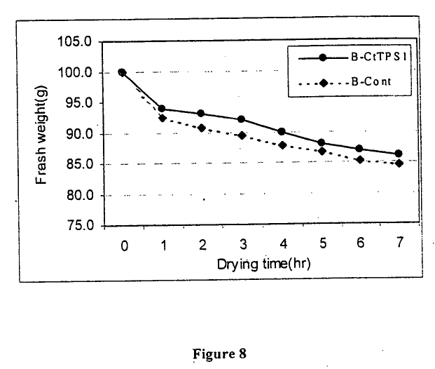

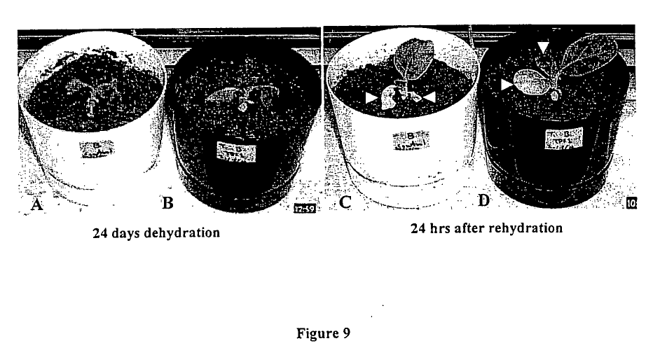

Mature leaves from fully-grown plants were tested for their ability to regulate water loss under drought conditions. When detached leaves were air dried, control and chloroplast transgenic plants lost water to the same extent (FIG. 8). Control and chloroplast transgenic potted plants were not watered for 24 days. Again, both showed dehydration to the same extent (FIGS. 9A,B). However, upon rehydration, fully dehydrated leaves (indicated by arrows, FIGS. 9C,D) recovered in chloroplast transgenic plants but not in controls.

This invention is exemplified by the following non-limiting example:

### EXAMPLE ONE

Plant, A. tumefaciens and E. coli culture: For transformation experiments, Nicotiana tabacum var. xanthi and Burley were grown in MS medium in the Magenta culture box (Sigma, USA). For drought tolerance assays of transgenic tobacco plants, the rooted young plants were transferred to pre-swollen Jiffy-7 peat pellets (Jiffy Products, Norway) inside the greenhouse. Plants used for enzyme assays were grown and kept in Magenta culture boxes. Seven or 8 leaf stage plants were used for enzyme assays. Two to three-week old young transgenic tobacco plants were used for stress analyses. (Agrobacterium tumefaciens strain LBA4404 was grown in the YEP medium at 29° C. In a shaking incubator. Other E. coli strains were cultured and maintained as described in Sambrook et al.

Plasmid construction and antibody production: For hyper-expression of the TPS1 in E. Coli for antibody production, the yeast TPS1 gene was cloned into plasmid pQE30 (Qiagen) and subsequently transformed into E. coli strain M15 [pREP4]. The resulting E. coli transformant was grown at 37° C. to an A₆₀₀ of 0.5-0.8 and induced by 2 mM isopropyl-β-D-thiogalactopyranoside (IPTG) for 1-5 hours. The induced cells were harvested and lysed by sonication. SDS-PAGE analysis showed the presence of TPS1 protein in crude cell extracts, even with Coomassie Blue stain, indicating high levels of expression. Western blot analysis using TPS1 antibody confirmed the true identity of the expressed protein (data not shown). The recombinant protein was purified using Ni²⁺ resin, using the procedures provided by the manufacturer. Affinity column purified recombinant protein was analyzed for purity by SDS-PAGE. Protein concentrations were determined using the Bio-Rad (USA) protein assay kit with BSA as a standard. Polyclonal antibody was generated using the purified TPS1 protein by the Takara Shuzo Co. (Japan).

Vector construction for plant transformation: The yeast 1.537 kbp TPS1 gene was inserted into the Xba1 site of pCt vector generating pCt-TPS1 (FIG. 2B). For the nuclear transformation, the yeast TPS1 gene was inserted into the pHGTPS1 vector in which the TPS1 gene is driven by the CaMV 35S promoter. The resulting vector confers hygromycin resistance because of the hygromycin phosphotransferase gene driven by the NOS promoter.

Chloroplast and nuclear transformation: For chloroplast transformation, particle bombardment was carried out using a helium driven particle gun, Biolistic PDH1000. Briefly, chloroplast vectors, pCt and pCt-TPS1 were delivered to tobacco leaves (Burley) using 0.6 μm gold microcarriers (Bio-11 Rad) at 1,100 psi with a target distance of 9 cm. For nuclear transformation, pHGTPS1 was mobilized into the A crobacterium tumefaciens strain LBA4404 by electroporation using Gene Pulsar (Bio-Rad. USA). The resulting Agrobacterium strain was used in leaf disc transformation of wild type N. tabacum var. xanthi.

Chloroplast DNA isolation and PCR: Total DNA was extracted from leaves of wild type and transformed plants using CTAB extraction buffer described. PCR was carried out to confirm spectinomycin resistant chloroplast tranformants using Peltier Thermal Cycler PTC-200 (MJ Research, USA). Three primer sets, 2P(5′-GCGCCTGACCCTG AGATGTGGATCAT-3′)-2M(5′-TGACTGCCCAACCTGAGAGCGGACA-3′), 3P(AAAACCCGTCCTCAGTTCGGATTGC)-3M(CCGCGTTGTTTCATCA AGCCTTACG) and −5P(CTGTAGAAGTCACCATTGTTGTGC), 5M(GTCCAAGAT AAGCCTGTCTAGCTTC) were used for the PCR. PCR reactions were carried out as described elsewhere (Daniell et al., 1998; Guda et al., 2000).

RNA isolation and Northern Slot analysis: Total RNA was extracted from transgenic tobacco plants using Tri Reagent (MRC, USA) following manufacturer's instruction. For northern blots, RNA samples (10 μg of total RNA per lane) were electrophoresed on a 1.5% agarose-MOPS gel containing formaldehyde. Uniform loading and integrity of RNAs were confirmed by examining the intensity of ethidium bromide bound ribosomal RNA bands under UV light. RNAs on the gel were transferred onto Hybond-N membrane (Amersham, USA). The membrane was hybridized to radiolabeled TPS1 probe and washed at 65° C. in a solution of 0.2×SSC and 0.1% SDS for 20 min twice. The blot was exposed to an X-ray film at −70° C. overnight. Transcripts were quantified using the BiolD++program with Vilber Lourmat Image Analyzer (Bioprofil, France).

Western Blot analysis: Tobacco total protein extracts were prepared by modified methods described by Ausubel et al. The total extracts were fractionated on a 10% one-dimensional SDS-PAGE, transferred to Biotrace PDVF nitrocellulose membrane (Gelman Sciences, USA), and immunostained using Renaissance Western Blot Chemiluminescence Reagent (NEN Life Science Products, USA) according to manufacturer's instructions. Each lane was loaded with 100 μg of total protein. The primary antibody used was anti-TPS1 at a 5000-fold dilution. The secondary antibody was anti-rabbit IgG HRP conjugate at a 2000-fold dilution (Promega, USA).

Drought tolerance and biochemical characterization: For analyses of drought tolerance, 2-3 week old transgenic tobacco plants were used. Seeds of chloroplast and nuclear tranformants were germinated on MS plates containing 3% or 6% PEG (MW 8,000). TPS1 enzyme assay was performed spectrophometrically by the method described by Londesbrough and Vuorio. For quantitative determination of T6P and trehalose, carbohydrates were extracted from aerial parts of transgenic or wild type tobacco plants by treatment in 85% ethanol at 60° C. for 1 hour. The amount of T6P and trehalose were measured by high-performance liquid chromatography (HPLC) on a Waters system equipped with a Waters High Performance Carbohydrate Column (4.6×250 mm) and a refractive index detector. The insoluble phase system was 75% acetanitrile-25% H₂O with a flow rate of 1.0 ml/min.

### References

Thevelein, J. M. & Hohmann, S. Trehalose synthase: guard to the gate of glycolysis in yeast? Trends in Bioscience. 20, 3-10 (1955).
Singer, M. A. & Lindquist, S. Thermotolerance in Saccharomyces cerevisiae: the Yin and Yang of trehalose. Trends in Biotech. 16, 460-468 (1998).
Elbein, A. D. The metabolism of a _-trehalose. Adv Carbohyd Chem Biochem. 30, 227-256(1974).
Mackenzie, K. F., Singh, K. K. & Brwon, A. D. Water stress plating hypersensitivity of yeast: protective role of trehalose in Saccharomyces cerevisiae. J Gen Microbial. 134, 1661-1666 (1988).
DeVigilio, C., Hottinger, T., Dominguez, J., Boller, T. & Wiekman, A. The role of trehalose synthesis for the acquisition of thermotolerance in yeast 1. Genetic evidence that trehalose is a thermoprotectant. Eur J. Biochem. 219, 179-186 (1994).
Sharma, S. C. A possible role of trehalose in osmotolerance & ethanol tolerance in Saccharomyces cerevisiae. Fems Microbiology Letters. 152, 11-15 (1997).
Crowe J. H., Hoekstra F. A. & Crowe L. M. Anhydrobiosys. Annu Rev Physiol. 54, 579-599 (1992).
Bianchi, G. Gamba, A. & Limiroli, R. The unusual sugar composition in leaves of the resurrection plant Myrotharnnus flatbellifolia. Physiol Plantarum. 87, 223-226 (1993).
Drennan, P. M., Smith, M. T., Goldsworthy, D. & Van Staden, J. The occurrence of trehalose in the leaves of the desiccation-tolerant angiosperm Myrothamnus flabellifolius Welw. J. Plant Physiol. 142, 493-496 (1993).
Colaco, C. Sen. S. Thangavelu, M., Pinder, S. & Roser, B. Extraordinary stability of enzymes dried in trehalose: simplified molecular biology. Biol technology, 10, 1007-1011(1992).
Iwahashi, H., Obuchi, K., Fujii, S. & Komatsu, Y. The correlative evidence suggesting that trehalose stabilizes membrane-structure in the yeast Saccharomyces cerevisiae. Cell. Mol. Biol. 41, 763-769 (1995).
Holmstrom, K. O., Mantyla, M., Wekin, B., Mandal, A., Palva. E. T., Tunnela, O. E. & Londesborough, J. Drought tolerance in tobacco. Nature. 379, 683-684 (1996).
Goddijyn, O. J. M., Verwoerd, T. C., Voogd, E., Krutwagen, W. H. H., ce Graff, P. T. H. M., Poels, J., van Dun, K., Ponstein, A. S., Damm, B. & Pen, K. Inhibition of trehalase activity enhances trehalose accumulation in transgenic plants. Plant Physiol. 113, 181-190 (1997).
Romero, C., Belles, J. M., Vaya, J. L., Serrano, R. & Culianz-Macia, F. A. Expression of the yeast trehalose-6-phosphate synthase gene in transgenic tobacco plants: pleiotropic phenotypes include drought tolerance. Planta. 201, 293-297 (1997).
Serrano, R., Culianz-Macia, F. A. & Moreno, V. Genetic engineering of salt & drought tolerance with yeast regulatory genes. Scientia Horticulture 78: 261-269.
During K. Hippe S. Kreuzaler F, Schell J (1990) Synthesis and self-assembly of a functional monocional antibody intransgenic Nicotianatabacum. Plant Molecular Biology. 15,281-293 (1999).
Daniell, H. & Guda, C. Biopolymer production in microorganisms and plants. Chemistry and industry. 14, 555-560 (1997).
Nuccio, M. L., Rhodes, D., McNeil, S. D. & Hanson, A. D. Metabolic engineering of plants for osmotic stress resistance. Crrunt opinion in plant biology 2: 128-134 (1999).
Daniell, H. New tools for chloroplast genetic engineering. Nat. Biotechnol. 17, 855-856. (1999).
Daniell, H. GM crops: Public perception and scientific solutions. Trends in Plant Science. 4,467-469 (1999).
Daniell, H. Environmentally friendly approaches to genetic engineering. In vitro Cellular and Developmental Biology-Plant. 35, 361-368 (1999).
Daniell, H. Genetically modified food crops: current concerns and solutions for the next generation crops. Biotechnology and Genetic Engineering Reviews, 17, in press.
Hoyle, B. Canadian farmers seek compensation for genetic pollution. Nat. Biotechnol 17, 747-748 (1999).
Finnegan J. & McElroy, D. Transgene Inactivation: Plants fight back. Biotechnology. 12, 883-888 (1994).
Kota, M., Daniell, H., Varma, S., Garcynski, F., Gould. F. & Morar, W. J. Overexpression of Bacillius thuringiensis Cry2A protein in chloroplasts confers resistance to plants against susceptible and Bt resistant insects. Proc Natl Acad Sci USA. 96, 1840-1845 (1999).
Daniell, H., Data, R., Varma, S., Gray, S.&Lee, S.-B. Containment of herbicide resistance through genetic engineering of the chloroplast genome. Nature Biotech. 16, 345-348. (1998).
Scott. S. E. & Wilkinson, M. J. Low probability of chloroplast movement from oilseed rape (Brassica napus) into wild Brassica napa. Nat. Biotechnol. 17: 390-392. (1999).
Brixey, P. J. Guda, C. & Daniell, H. The chloroplast psbA promoter is more efficient in E. coli than the T7 promoter for hyper-expression of a foreign protein. Biotechnology letters. 19, 2395-399 (1997).
Guda, G., Lee, S-B., & Daniell, H. Stable expression of a biodegradable protein-based polymer in tobacco chloroplasts. Plant Cell Reports 18, (1999).
Daniell, H. Transformation and foreign gene expression in plants mediated by microprojectile bombardment. Meth. Mol. Biol. 62, 453-488 (1997).
Roser, B. & Colaco, C. A sweeter way to fresher food New Scientist. 138, 24-28 (1993).
Sambrook. J., Maniatis, T. & Fritsch, E. J. Molecular Cloning: A Laboratory Manual. Cold Spring Harbor Laboratory, Cold Spring Harbor, N.Y. (1989).
Dellaporta, S. L., Wood, J. & Hicks, J. B. A plant DNA minipreparation: Version II. Plant Mol Biol Rep. 1, 19. (1983).
Ausubel, F., Brent, R., Kingston, R. E., Moore, D. D., Sediman, J. G., Smith, J. A. & Struhl, K. Short protocols in molecular biology. Wiley and Sons, Inc. USA (1995).
Londesbrough, J. & Vuorio, O. Trehalose-6 phosphate synthase/phosphat-ase complex from baker's yeast: purification of a protelytically activated form. J. Gen. Microbiology. 137, 323-330 (1991).
Herbicide Resistance Crops Agricultural Environmental Economic Regulatory and Technical Aspects, Duke, S. O., edt., CRC Press, Inc. (1996).
Herbicide Resistance in Plants, Biology and Biochemistry, Powles, S. B., and Holtum, J. A. M., eds., CRC Press, Inc. (1994).
Peptides: Design, Synthesis and Biological Activity, Basava, C. and Anantharamaiah, G. M., eds., Birkhauser Boston, 1994.
Protein Folding: Deciphering the Second Half of the Genetic Code, Gierasch, L. M., and King, J., eds., American Association For the Advancement of Science (1990).

1. An integration and expression plastid vector competent for stably transforming the plastid genome of which confer stress tolerance which comprises an expression cassette which comprises as operably joined components, a 5′ part of the plastid DNA sequence inclusive of a spacer sequence, a promoter operative in said plastid, a selectable marker sequence, a DNA sequence encoding for an osmoprotectant, at least one restriction site for the insertion of a heterologous target DNA sequence, a transcription termination region functional in said plastid, and the 3′ part of the plastid DNA sequence inclusive of a spacer sequence.

2. The vector of claim 1 further comprising a heterologous DNA sequence which codes for a molecule of interest that is inserted in one of the restriction sites.

3. The vector of claim 2 where the molecule of interest is a polypeptide.

4. A vector of claim 2 or 3, wherein said vector further comprises a ribosome binding site and a 5′ untranslated region (5′ UTR) to enhance expression.

5. A vector of claim 2, 3, or 4 wherein the osmoprotectant is selected from a group consisting of sugars, sugar alcohols, sugar derivates, and amino acids including proline and glycine-betaine.

6. A vector of claim 5 wherein the osmoprotectant is trehalose.

7. A vector of claim 5 wherein the trehalose is at least one of the complex TPS1, TPS2, TPS3 or TSL1.

8. The vector of claim 2, 3 or 4 wherein the osmoprotectant is selected from a group consisting of TSP 1, E. Coli otsA, stachyose, and ononitol.

9. The vector of claim 5 wherein the osmoprotectant is a sugar.

10. The vector of claim 9, wherein the sugar is a monosacharide including but not limited to fructose.

11. The vector of claim 9, wherein the sugar is a disaccharide including but not limited to sucrose.

12. The vector of claim 9, wherein the sugar is a trisaccharide including but not limited to raffinose.

13. The vector of claim 9 wherein the sugar is dulcitol.

14. The vector of claim 5 wherein the osmoprotectant is a sugar alcohol.

15. The vector of claim 14 wherein the sugar alcohol is a polyhyric alcohol.

16. The vector of claim 15 wherein the polyhyric alcohol is a trihydric alcohol including but not limited to glucoglycerol.

17. The vector of claim 15 wherein the polyhyric alcohol is a tetrahydric alcohol including but not limited to erythritol.

18. The vector of claim 15 wherein the polyhyric alcohol is a hexahydric alcohol including but not limited to mannitol or sorbitol.

19. A vector of claim 2, 3 or 4 wherein at least one DNA encodes a component of trehalose synthase that is under the control of a promoter to produce a transgenic plant.

20. The vector of claim 19 wherein the promoter is constitutive.

21. The vector of claim 19 wherein the promoter is tissue specific, light-induced, or stress-induced.

22. A stably transformed plant which has been transformed by the vector of any one of claims 2-21, wherein the transformed plant is more tolerant of stresses selected from a group consisting of water-deprivation, freezing, salt, heat and cold than is the untransformed plant.

23. The plant of claim 22 wherein the plant does not include target DNA.

24. A stably transformed plant of claim 22, or the progeny thereof including seeds, wherein said plant display no negative pleiotropic effects.

25. A transgenic plant of any one of claims 22-25, wherein the plant is a transgenic plant which is morphologically indistinguishable from an untransformed plant.

26. A transgenic plant of any one of claims 22-25, wherein the plant is a solanaceous plant edible for a mammal.

27. A transgenic plant of any one of claims 22-25, wherein the plant is a crop plant edible for a mammal.

28. A transgenic plant of either claim 26 or 27, wherein the mammal is a human.

29. A transgenic plant of any one of claims 22-25, wherein the plant is a monocotyledonous plant selected from the group of rice, wheat, grass, rye, barley, oat, or maize.

30. A transgenic plant of any one of claims 22-25, wherein the plant is a dicotyledonous plant selected from the group of soybean, peanut, grape, sweet potato, pea, canola, tobacco, tomato or cotton.

31. A transgenic plant of any one of claims 22-25, wherein the plant is tobacco, tomato, potato, rice, brassica, cotton, maize or soybean.

32. A method of conferring drought resistance to plants, said method comprising introducing into the plastid of plant species that are susceptible to water stress, an expression cassette which comprises as operably joined components; a 5′ part of the plastid DNA sequence inclusive of a spacer sequence, a promoter operative in said plastid, a DNA sequence encoding a gene which confers osmoprotection, a heterologous DNA sequence encoding a molecule of interest, a selectable marker sequence, a transcription termination region functional in said plastid, and a 3′ part of the plastid DNA sequence inclusive of a spacer sequence.

33. The method of claim 32, wherein said method further comprises culturing said plant in a plant growth medium containing an effective amount of polyethylene glycol (PEG) for selection, and selecting transformed plant cells capable of growth in said plant growth medium.

34. The method of claim 33, wherein said method further comprises regenerating the selected transformed plant cells into stable transgenic plants.

35. A method of increasing trehalose accumulation in plant cells thereby conferring osmotic stress resistance to said plant cells, where said method comprises introducing to the plastid of plant species that are susceptible to osmotic stress an expression cassette which comprises as operably joined components, a 5′ part of the plastid DNA sequence inclusive of a spacer sequence, a promoter operative in said plastid, a DNA sequence encoding the Yeast T6P synthase (TSP) gene which confers drought resistance, a heterologous DNA sequence encoding a molecule of interest, a selectable marker sequence, a transcription termination region functional in said plastid, and a 3′part of the plastid DNA sequence inclusive of a spacer sequence.

36. The method of claim 35, wherein said method further comprises culturing said plant in a plant growth medium containing an effective amount of polyethylene glycol (PEG) for selection, and selecting transformed plant cells capable of growth in said plant growth medium.

37. The method of claim 36, wherein said method further comprises regenerating the selected transformed plant cells into stable transgenic plants.

38. The vector of any one of claims 1-21, wherein said plastid is a chloroplast.

39. The vector of claim 38, wherein the vector is a universal chloroplast vector.

40. The methods of any one of claims 32-37, wherein the plastid is a chloroplast.


SEQUENCE LISTING (6 sequences)

SEQ ID NO: 1 - DNA, 26 bp, Artificial Sequence
  Primer
gcgcctgaccctgagatgtggatcat

SEQ ID NO: 2 - DNA, 25 bp, Artificial Sequence
  Primer
tgactgcccaacctgagagcggaca

SEQ ID NO: 3 - DNA, 25 bp, Artificial Sequence
  Primer
aaaacccgtcctcagttcggattgc

SEQ ID NO: 4 - DNA, 25 bp, Artificial Sequence
  Primer
ccgcgttgtttcatcaagccttacg

SEQ ID NO: 5 - DNA, 24 bp, Artificial Sequence
  Primer
ctgtagaagtcaccattgttgtgc

SEQ ID NO: 6 - DNA, 25 bp, Artificial Sequence
  Primer
gtccaagataagcctgtctagcttc